(19) United States
(12) Patent Application Publication
(10) Pub. No.: US 2025/0102525 A1
(43) Pub. Date: Mar. 27, 2025

(54) Device for Patient-Specific Prognosis

(71) Applicant: Creighton University, Omaha, NE (US)

(72) Inventors: Andrew Edet Ekpenyong, Omaha, NE (US); Chisom Nwakama, Marshall, MN (US)

(21) Appl. No.: 18/891,539

(22) Filed: Sep. 20, 2024

Related U.S. Application Data
(63) Continuation of application No. 63/584,334, filed on Sep. 21, 2023.

(51) Int. Cl. G01N 33/80; B01L 3/00

(52) U.S. Cl. CPC G01N 33/80; B01L 3/502761; B01L 2200/0652; B01L 2200/16; B01L 2300/0654; B01L 2300/0883; G01N 2800/22

(57) ABSTRACT

Systems and methods for providing patient-specific prognoses of blood conditions utilizing a microfluidic microcirculation mimetic (MMM) are described. In an aspect, an MMM device includes, but is not limited to, an inlet adhesion area configured to receive the blood sample; a microfluidic channel fluidically coupled with the inlet adhesion area to receive the blood sample from the inlet adhesion area; and an outlet adhesion area fluidically coupled with an outlet end of the microfluidic channel to receive the blood sample from the microfluidic channel, wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells.

### CROSS-REFERENCE TO RELATED APPLICATIONS

The present application claims the benefit of 35 U.S.C. § 119(e) of U.S. Provisional Application Ser. No. 63/584,334, filed Sep. 21, 2023, and titled “DEVICE FOR PATIENT-SPECIFIC PROGNOSIS.” U.S. Provisional Application Ser. No. 63/584,334 is herein incorporated by reference in its entirety.

### BACKGROUND

Sickle cell disease (SCD) refers to a group of blood disorders that can result in a variety of health conditions or issues for individuals affected with SCD. One example health condition includes vaso-occlusive crises (VOC) in which deformations of sickle-shaped red blood cells obstruct blood vessels, such as capillaries, and can lead to organ necrosis or failure, pain, ischemia, or other conditions.

### SUMMARY

Systems and methods for providing patient-specific prognoses of blood conditions utilizing a microfluidic microcirculation mimetic (MMM) are described. In an aspect, a system for patient-specific prognosis of sickle cell disease (SCD) includes, but is not limited to, a microfluidic microcirculation mimetic (MMM) device configured to pass a blood sample therethrough, the MMM device including an inlet adhesion area configured to receive the blood sample, a microfluidic channel fluidically coupled with the inlet adhesion area to receive the blood sample from the inlet adhesion area, and an outlet adhesion area fluidically coupled with an outlet end of the microfluidic channel to receive the blood sample from the microfluidic channel; a sample camera mounted relative to the MMM device, the sample camera configured to record images of the blood sample as the blood sample at least one of passes through the MMM device or is retained in the MMM device; a pump configured to introduce the blood sample to the inlet adhesion area and to transfer the blood sample through the MMM device; and a controller communicatively coupled with the pump, the controller configured to control a pump rate of the pump to establish a flow rate of the blood sample through the MMM device, wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells.

In an aspect, a microfluidic microcirculation mimetic (MMM) device configured to receive a blood sample for patient-specific prognoses of blood conditions in the blood sample includes, but is not limited to, an inlet adhesion area configured to receive the blood sample; a microfluidic channel fluidically coupled with the inlet adhesion area to receive the blood sample from the inlet adhesion area; and an outlet adhesion area fluidically coupled with an outlet end of the microfluidic channel to receive the blood sample from the microfluidic channel, wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells.

In an aspect, a method for determining a cell detachment force of a red blood cell from a blood sample for providing patient-specific prognoses of blood conditions in the blood sample includes, but is not limited to, introducing a blood sample at a first flow rate to a microfluidic microcirculation mimetic (MMM) device, the MMM device including an inlet adhesion area configured to receive the blood sample a microfluidic channel fluidically coupled with the inlet adhesion area to receive the blood sample from the inlet adhesion area, and an outlet adhesion area fluidically coupled with an outlet end of the microfluidic channel to receive the blood sample from the microfluidic channel, wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells; ceasing flow of the blood sample to the MMM device to permit attachment of blood cells from the blood sample onto one or more internal surfaces of the MMM device; incrementally increasing the flow rate of the blood sample to detach cells from the one or more internal surfaces; and determining a detachment force associated with detachment of the cells from the one or more internal surfaces based on the flow rate at a time at which one or more of the cells detached from the one or more internal surfaces.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. In the figures, the use of the same reference numbers in different instances in the description and the figures may indicate similar or identical items.

FIG. 1 is a schematic diagram of a microfluidic microcirculation mimetic (MMM) device in accordance with example embodiments of the present disclosure.

FIG. 2A is a phase contrast microscope image of an MMM device, with a magnification of a serpentine section of the MMM device shown, in accordance with example embodiments of the present disclosure.

FIG. 2B is a phase contrast microscope image of the serpentine section of the MMM device shown having constricted sections, in accordance with example embodiments of the present disclosure.

FIG. 3 is a schematic diagram of a system for providing patient-specific prognoses of blood conditions utilizing an MMM device, in accordance with example embodiments of the present disclosure.

FIG. 4A is a phase contrast microscope image of cell adhesion of cells in the inlet adhesion area of an MMM device, in accordance with example embodiments of the present disclosure.

FIG. 4B is a phase contrast microscope image of cell adhesion of cells in the outlet adhesion area of an MMM device, in accordance with example embodiments of the present disclosure.

FIG. 5 is an image of morphometry results of circularity of adherent cells present in an MMM device, in accordance with example embodiments of the present disclosure.

FIG. 6 is a schematic diagram of the system of FIG. 3, shown with a valve system for controlling gas conditions within the MMM device and with a bioreaction chamber for interacting multiple cell types prior to introduction to the MMM device, in accordance with example embodiments of the present disclosure.

### DETAILED DESCRIPTION

### Overview

Determining patient prognoses and appropriate treatment plans for individuals with blood diseases or disorders, such as sickle cell disease (SCD), can be a challenging prospect, as treatments suitable for one patient might be less effective or ineffective for another patient having the same blood condition. To assist with patient-specific research and monitoring, lab-on-chip microfluidic devices have emerged as advanced research platforms for basic and clinical research applications. In particular, microfluidic microcirculation mimetic (MMM) devices have been developed to provide point of care (POC) diagnostic tools and patient monitoring systems by mimicking conditions found in the body. For example, MMM devices can be utilized to mimic conditions in microcapillaries found during pulmonary microcirculation, deformability of white blood cells in the context of chronic occlusive pulmonary disorder (COPD), translocation events of cancer metastasis, and other biological environments and microfluidic conditions.

In other aspects, MMM devices can be utilized for near in-vivo visualization of vaso-occlusive crises (VOC) in patients with sickle cell diseases. However, prognostic visualization of conditions of SCD remains critical due in part to SCD remaining a highly-studied genetic disorder, but with the phenotypic manifestations in patients differing widely. For instance, patients taking the same medication (e.g., an FDA-approved drug, such as hydroxyurea) show different responses and outcomes. Additionally, use of MMM devices have shown that the transit time for red blood cells (RBC) through microfluidic passages can vary between different patients and that patient blood samples can provide dynamic changes in percentage of sickled RBC.

Accordingly, the present disclosure is directed, at least in part, to systems and methods for providing patient-specific prognoses of blood conditions utilizing an MMM device. The MMM device includes an inlet adhesion area configured to quantify cell adhesion prior to introduction to an unbranched microfluidic channel and an outlet adhesion area configured to quantify cell adhesion following passage of cells from the unbranched microfluidic channel. The microfluidic channel is generally formed from a serpentine structure to facilitate use on a lab-on-chip structure for point of care (POC) diagnostics and patient monitoring with near in-vivo visualization. The microfluidic channel can have a common cross-sectional area such that no constrictions are present. Alternatively or additionally, the microfluidic channel can include one or more constrictions present to visibly deform the cell as the cell passes through the constriction. In aspects, the inlet adhesion area and the outlet adhesion area includes portions that are functionalizable adhesion areas to alter the adhesiveness of RBCs, other blood components, and other cells in the respective areas. The MMM device facilitates rapid, quantitative, and visual readouts which correlate with efficacy of drugs and other variables for the improved management of SCD patients. For instance, the MMM device can facilitate the determination of cell adhesiveness to VOC in a manner distinct from the contribution of cell deformability to account for differences in the way that blood conditions, such as SCD, manifest in the blood samples of patients.

A pump system (e.g., a syringe pump) is utilized to control the flow of the sample through the MMM device. In implementations, the pump is controlled to facilitate determination of cell detachment force to provide a robust quantification of cell attachment. For instance, the pump can initially operate to introduce a sample of cells through the MMM device and into the outlet cell attachment area. The pump can less cease operation or otherwise reduce the flow rate of the sample to permit a portion of the cells to attach. Following attachment, the pump can be reactivated or otherwise operated to increase the pump flow rate (e.g., increase the flow rate of the sample gradually over time) to observe when individual cells or groups thereof detach from the outlet cell attachment area, which in turn can facilitate determination of cell detachment force based on the shear forces experienced by the cell(s) at the flow rate under which the cell(s) detached.

In aspects, the MMM device is part of a system that includes a valve system to control oxygen parameters or other gaseous parameters to analyze individual patient blood samples according to a variety of oxygen environments (e.g., hypoxic, normoxic, hyperoxic etc.) or other gaseous environments, including transitions from one environmental state to another. In aspects, the system can include a bioreaction chamber to permit interaction between RBCs and other cells, drugs, chemicals, or biological components to visualize and/or quantify the respective interaction and the effects thereof.

### Example Implementations

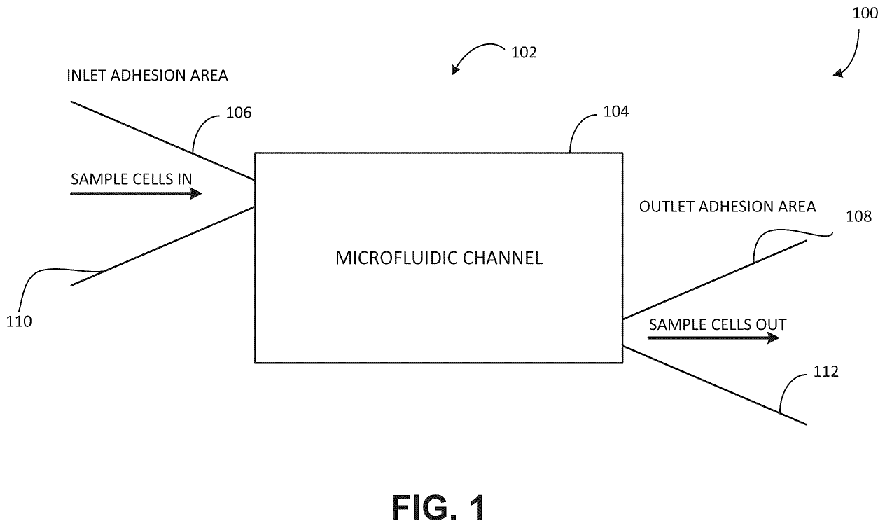

Referring to FIGS. 1-6, a system for providing patient-specific prognoses of blood conditions utilizing an MMM device (“system 100”) are shown. In general, the system 100 includes an MMM device 102 including a microfluidic channel 104 through which a patient blood sample can pass, an inlet adhesion area 106 fluidically coupled with an inlet end of the microfluidic channel 104, and an outlet adhesion area 108 fluidically coupled with an outlet end of the microfluidic channel 104. In implementations, the microfluidic channel 104 is formed as a serpentine fluid channel having a plurality of alternating fluid paths from a single fluid channel without branches from the fluid path. The microfluidic channel 104 can have dimensions based on the type of sample that is to be analyzed with the MMM device 102. For example, for RBC analysis, the microfluidic channel 104 can have an internal cross-sectional width from about 10 μm to about 30 μm. For example, the internal cross-sectional width of the microfluidic channel 104 can be about 15 μm. Similarly, for RBC analysis, the microfluidic channel 104 can have an internal cross-sectional height from about 10 μm to about 30 μm. For example, the internal cross-sectional height of the microfluidic channel 104 can be about 15 μm. The length of the microfluidic channel 104 can be dependent on the type of sample that is to be analyzed with the MMM device 102, a type of pump used in the system 100, the overall size footprint of a fluid on chip device (FOC) on which the MMM device 102 is configured, a size of a microscope stage used to view the flow of sample through the MMM device 102, or the like. In implementations, the inlet adhesion area 106 and the outlet adhesion area 108 are substantially larger than the microfluidic channel 104. For example, implementations, the length of the inlet is about four times the length of the microfluidic channel 104, with each having a substantially constant depth. The proportions of length to depth can provide visualization of the MMM device 102 and the samples interacting therewith utilizing a microscope with 4 times or 5 times magnification. In implementations, the inlet adhesion area 106 and the outlet adhesion area 108 are tapered relative to the inlet and outlet of the microfluidic channel 104. For instance, the tapering of the inlet adhesion area 106 can permit sample cells to be introduced to the MMM device 102 at relatively low flow rates until the sample cells reach the microfluidic channel 104.

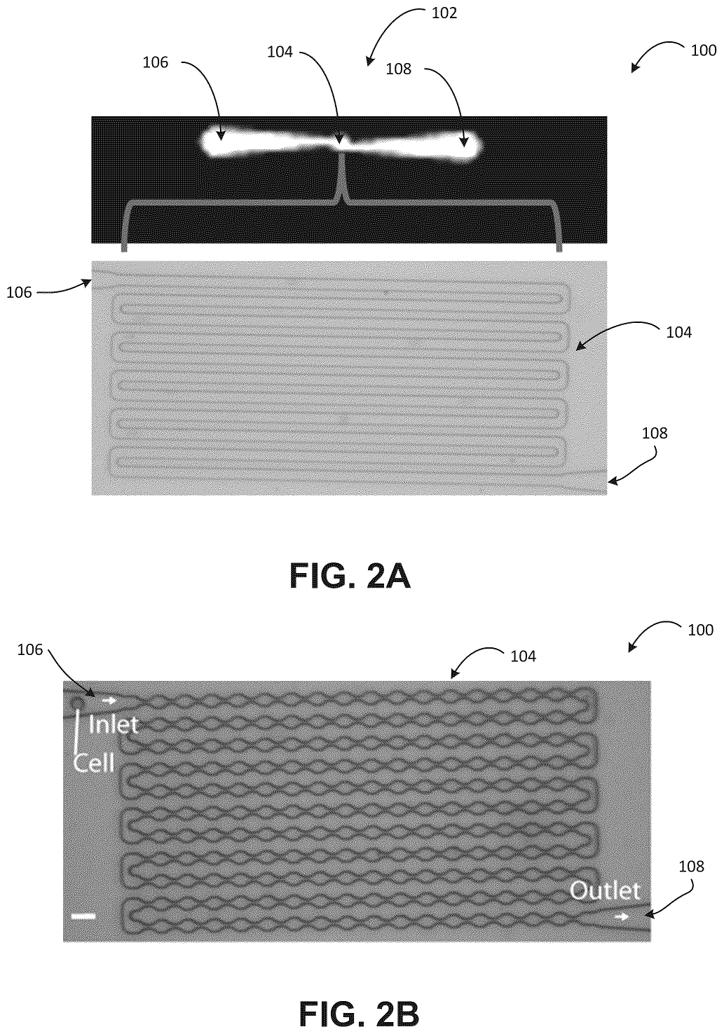

The microfluidic channel 104 can be formed from a fluid passageway having no constrictions such that the fluid passageway has a substantially consistent cross-sectional area from the inlet to the outlet such that no cross-sectional constrictions are present that could deform a blood cell from the patient blood sample. For example, the microfluidic channel 104 can include, but is not limited to, a substantially constant 15 μm height and width from the inlet to the outlet. An example of the microfluidic channel 104 having no constrictions is shown in FIG. 2A. Alternatively, the microfluidic channel 104 can be formed from a fluid passageway having one or more constrictions such that the fluid passageway includes one or more cross-sectional areas that constrict from a first wider cross-sectional area to a second constricted cross-sectional area. The constrictions are present to induce a deformation a blood cell from the patient blood sample as the blood cell passes through the construction. For example, for a microfluidic channel 104 can have a first height and width of 15 μm, a constriction in one or both of the height and the width can include, but is not limited to, a height and/or width of 9 μm, a height and/or width of 7 μm, a height and/or width of 5 μm, or the like. An example of the microfluidic channel 104 having constrictions is shown in FIG. 2B, where an example implementation of 187 constrictions is presented.

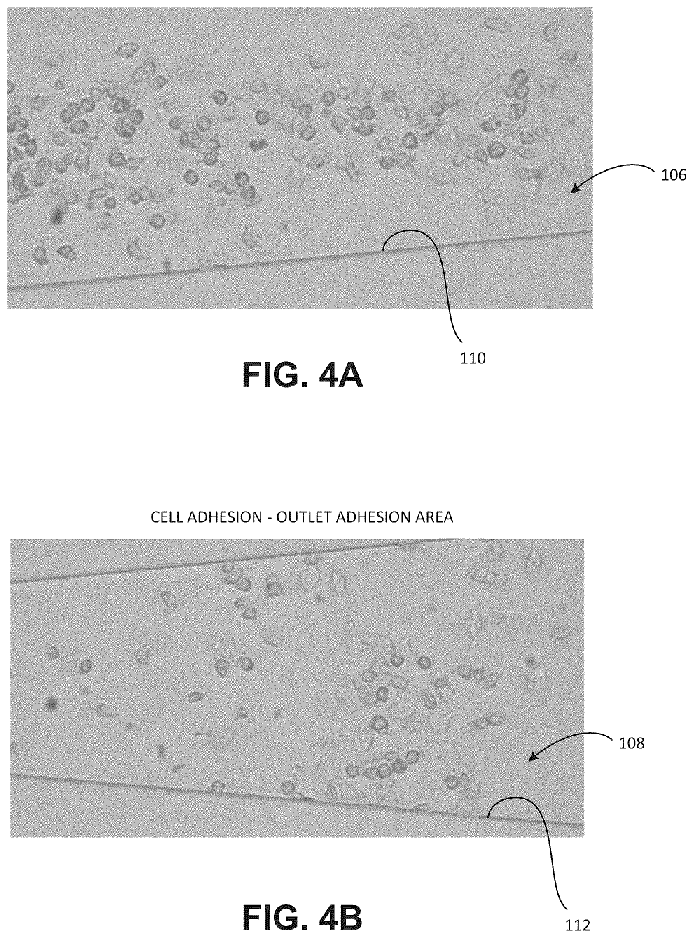

One or more portions of the MMM device 102 can include surfaces with predefined or adjustable functionalization to interact with portions of the sample (e.g., RBCs), such as to influence adherent cells to a greater extent than non-adherent or normal RBCs. Different functionalizations can compare how a particular cells type reacts or operates under various treatment conditions. For instance, one or more of the inlet adhesion area 106, the outlet adhesion area 108, or the microfluidic channel 104 can include functionalizable adhesion areas configured to influence the retention of adherent cells to the functionalizable adhesion areas. For example, referring to FIGS. 1, 4A, and 4B, the inlet adhesion area 106 is shown having an inlet functionalizable adhesion area 110 and the outlet adhesion area 108 is shown having an outlet functionalizable adhesion area 112, where the inlet functionalizable adhesion area 110 and the outlet functionalizable adhesion area 112 are structured on an interior surface of the respective adhesion areas. In implementations, the functionalizable adhesion areas are functionalized through addition of one or more surface treatments of chemicals or cells. For example, the chemicals or cells can include, but are not limited to, poly-D-lysine, poly-L-lysine, endothelial cell layer(s), hyaluronic acid, polyacrylamide gel, extracellular matrix protein (e.g., including glycoproteins, such as fibronectin), hydrogels providing heterogenous extracellular matrix proteins, collagen-derived extracellular matrix proteins, cell-secreted or cell-associated matrix-metallopreteases, or the like, or combinations thereof.

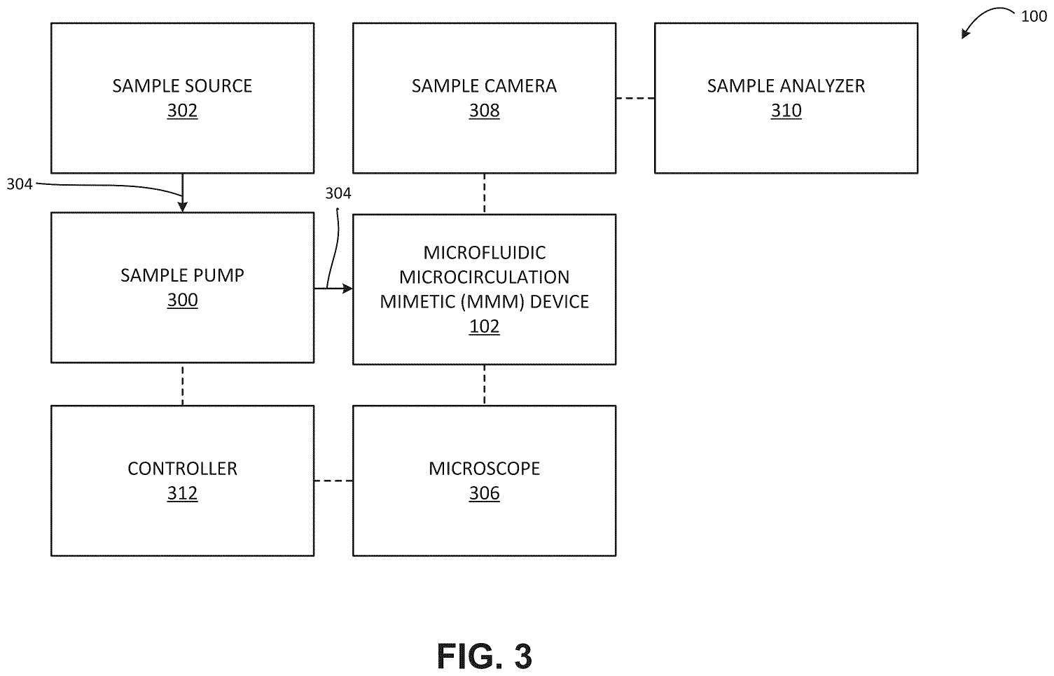

The MMM device 102 can facilitate determination of cell adhesion indices to quantify comparative cell adhesion. For instance, the system 100 can determine a pre-constriction adhesion index for cells in the inlet adhesion area 106 (e.g., for MMM devices 102 having a microfluidic channel 104 with constrictions), a post-constriction adhesion index in the outlet adhesion area 108 (e.g., for MMM devices 102 having a microfluidic channel 104 with constrictions), a constriction-independent inlet cell adhesion index in the inlet adhesion area 106 (e.g., for MMM devices 102 having a microfluidic channel 104 without constrictions), and/or a constriction-independent outlet cell adhesion index in the outlet adhesion area 108 (e.g., for MMM devices 102 having a microfluidic channel 104 without constrictions). In implementations, determination of the number of cells adhered or introduced is facilitated by a sample camera, such as by sample camera 308 described herein with reference to FIG. 3. In implementations, the cell adhesion index is determined as a ratio of the number of cells adhered within the inlet adhesion area 106 or the outlet adhesion area 108 to the number of cells introduced to the respective inlet adhesion area 106 or the outlet adhesion area 108. For example, the cell adhesion index can be determined in terms of physical variables and fluid mechanical principles associated with flow of sample through the MMM device 102, such as according to equations (1) through (4):

$$Q = \frac{V}{t} \qquad ( 1 )$$

$$σ = \frac{F}{A} \qquad ( 2 )$$

$$η is proportional to \frac{G}{σ v} \qquad ( 3 )$$

$$Q = \frac{π {r}^{4}}{8 η L} \times Δ P \qquad ( 4 )$$

where equation (1) provides the volumetric flowrate, Q, where V is the volume, and t is the time, equation (2) provides the shear stress experienced by the cells, o, where F is the tangential force, and A is the surface area, equation (3) provides a relationship for viscosity of the cell medium fluid, n, where G is a geometric factor dependent on the inlet adhesion area 106 (for inlet adhesion indices) or on outlet adhesion area 108 (for outlet adhesion indices), and v is the velocity of the cell medium, and equation (4) is Poiseuille's equation that links equations (1)-(3). In implementations, a known volumetric flow rate Q is applied (e.g., 99.9 μL/hr), where the inlet adhesion area 106 and the outlet adhesion area 108 have known surface areas, A. For cell adhesion index measurements, the cells are permitted to adhere to the inlet adhesion area 106 and the outlet adhesion area 108 following initial introduction, such as for 10 to 30 minutes, depending on the cell type. The flow rate during this adherent phase is zero. The flow rate Q is then increased until approximately 90% of the cells are detached or otherwise pulled away from the respective surface of the inlet adhesion area 106 and the outlet adhesion area 108. Since the pressure difference ΔP is proportional to the shear stress experienced by the cells, o, via equations (1)-(4), the cell adhesion index measurement can be obtained as a ratio of the flow rate when cells are adherent without detachment (e.g., when less than 10% are attached) and the flow rate when over 90% are detached. The cell adhesion index measurement can therefore provide a normalized readout of cell adhesion forces.

The cell adhesion index measurements can provide molecular level information about cell physiology and pathology, as appropriate, for a given sample. For instance, cell adhesion is a property related to cell communication and regulation, and is of fundamental importance in the development and maintenance of tissues. The mechanical interactions between a cell and its extracellular matrix (ECM) can influence and control cell behavior and function. The cell adhesion index facilitates quantification of a physiological event that happens in the body, for instance during blood flow, intravasation and extravasation of immune cells in and out of the circulatory system to carry out their functions including fighting infections, wound healing, clearance of dead cells, etc. For example, the cell adhesion index can facilitates direct and indirect quantification of effects of drugs on cells, effects of extracellular matrix via functionalization of the inlet/outlet, level of pathology (e.g., for ex vivo assessment of patient samples), factors important for pathogenesis, and combinations thereof, due to the physiological importance of cell adhesion.

Referring to FIG. 3, the system 100 is shown with the MMM device 102 with additional system components to provide patient-specific prognoses of blood conditions in accordance with example implementations of the present disclosure. The system 100 is shown generally including the MMM device 102 fluidically coupled with a sample pump 300 that is fluidically coupled with a sample source 302 (e.g., via one or more fluid lines 304). The sample pump 300 can include any pump suitable to transfer biological fluid samples and can include, but is not limited to, a syringe pump, a peristaltic pump, piezoelectric micropumps, or the like, or combinations thereof. The sample source 302 can include any container, fluid line, or the like suitable for holding a volume of a biological fluid sample (e.g., a blood sample) for analysis by the system 100. In implementations, the sample source 302 includes a biological sample autosampler having wells, vials, trays, or the like, of multiple fluid samples for serial analysis by the system 100. The sample pump 300 is operable to draw or push a sample received from the sample source 302 and transfer the sample to the inlet adhesion area 106 of the MMM device 102 (e.g., via one or more fluid lines 304). The sample pump 300 can also transfer the sample from the inlet adhesion area 106, through the microfluidic channel 104, to the outlet adhesion area 108.

The system 100 further includes a microscope 306 and a sample camera 308 to facilitate viewing and recording data associated with the sample passing through the MMM device 102 including, but not limited to, sample adhered to the inlet adhesion area 106, sample adhered to the outlet adhesion area 108, sample detached from the inlet adhesion area 106, sample detached from the outlet adhesion area 108, sample transit time through the MMM device 102, sample deformation through one or more constrictions of the microfluidic channel 104, or other aspects of operation of the system 100. In an implementation, the microscope 306 includes a phase contrast microscope to assist in view individual blood cells or other sample components as they interact with portions of the MMM device 102 or otherwise travel through the MMM device 102. The sample camera 308 can include any suitable image capture device to generate visual data for analysis by the system 100. For example, the sample camera 308 can include, but is not limited to, a charge-coupled device (CCD) camera. The sample camera 308 is communicatively coupled with a sample analyzer 310 to receive and process the image data generated by the sample camera 308 for determination of one or more characteristics of the sample including, but not limited to, pre-constriction adhesion index, post-constriction adhesion index, constriction-independent inlet cell adhesion index, constriction-independent outlet cell adhesion index, circularity, and cell detachment force, as described herein.

Operation of one or more components of the system 100 can be controlled by a computer controller 312 to coordinate operation of the system 100 to facilitate analysis of the sample. For example, the controller 312 can be communicatively coupled with the sample pump 300 to send control signals to the sample pump 300 to control a flow rate or pump speed of the sample pump 300. For instance, control of the flow rate or pump speed influences the rate at which the sample flows through the MMM device 102, where the sample camera 308 can capture the passage of individual blood cells or other sample components through or adhered onto portions of the MMM device 102. In implementations, the controller 312 changes the flow rate or pump speed of the sample pump 300 over time (e.g., increases over time, decreases over time, or combinations thereof) such that a single sample or a fluid acting on the sample is introduced at multiple flow rates, is permitted to rest within the MMM device 102 (e.g., with the sample pump 300 paused or otherwise inactivated), or combinations thereof.

In implementations, the controller 312 coordinates operation of the sample pump 300 to facilitate determination of cell detachment force by the system 100. Cell detachment force refers to the force required to detach adherent cells that are adhered to one or more surfaces of the MMM device 102 including, but not limited to, the inlet adhesion area 106, the microfluidic channel 104, the outlet adhesion area 108, a functionalized surface of the inlet adhesion area 106, the microfluidic channel 104, or the outlet adhesion area 108, or combinations thereof. In implementations, the cell detachment force is provided through fluid shear stress on the cells produced through flow rate control of the fluid by the sample pump 300. For instance, sample camera 308 can record the flow of cells, attachment of cells, and subsequent detachment of cells over time, where the sample analyzer can assign a time of cell detachment (e.g., of an individual cell, a group of cells, etc.) with a flow rate of the sample pump 300 at the time of cell detachment to calculate a corresponding cell detachment force.

In implementations, the system 100 coordinates the determination of cell detachment force by initially operating the sample pump 300 (e.g., via control signals from the controller 312) to introduce sample cells into the MMM device 102. The sample pump 300 then ceases operation to permit attachment of cells to one or more internal surfaces of the MMM device 102. The controller 312 then incrementally and/or gradually resumes flow of fluid through the MMM device 102 until a portion of cells, a majority of cells, or all the cells detach from their respective surfaces. During experimental analysis of an MMM device 102 having a 15 μm×15 μm internal cross section, most cells experienced detachment by a sample flow rate of 99.99 μL/hr, however detachment force can vary depending on the particular cell population, sample source, and the like. An average of the cell detachment force of a given sample can provide a robust quantification of cell adhesion that is independent of comparative cell adhesion indices.

Cell detachment force can be calculated based on shear stress (F/A) being proportional to the volumetric flowrate and the dynamic viscosity of the sample cell medium. For example, in implementations, the detachment force can be obtained through equation (5):

$$F / A = η ( channel ) Q \qquad ( 5 )$$

where F is the cell detachment force, A is the cross-sectional area of the MMM device 102 on which the cell is located, η is the dynamic viscosity of the cell medium, (channel) is a factor dependent on the MMM channel (determined experimentally), and Q is the volumetric flowrate of the cell medium, in furtherance of equations (1) through (4) provided herein.

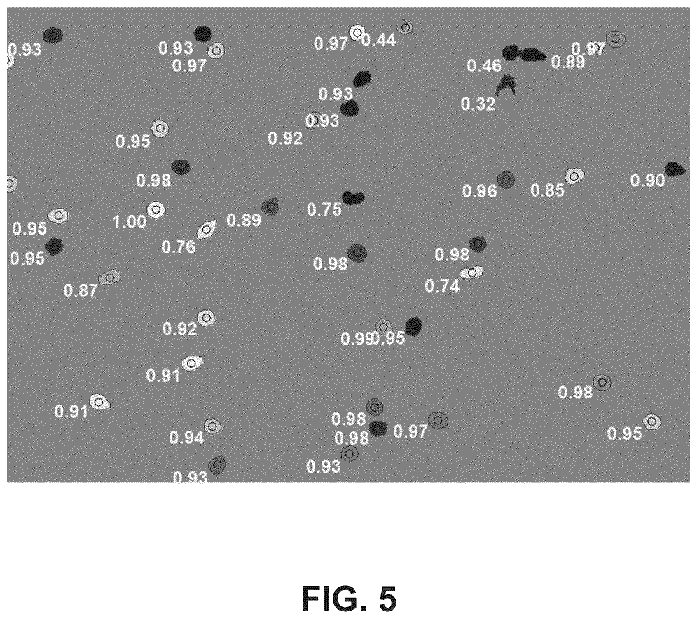

Referring to FIG. 5, the system 100 can facilitate determination of circularity of individual blood cells of a particular sample, which can provide morphometry-based selection of adherent cells for measurement of cell adhesion at the single cell level. For instance, the sample analyzer 310 can calculate a circularity value for individual blood cell images from a sample fluid flowing through or otherwise interacting with the MMM device 102 captured by the sample camera 308. In implementations, circularity, C, examples of which are shown in FIG. 5, is calculated according to equation (6):

$$C = ( 4 π A ) / ( {P}^{2} ) \qquad ( 6 )$$

where A is the area of the measured object (e.g., cell area) and P is the perimeter of the object (e.g., cell perimeter), ranging from 0 for infinitely elongated polygon (e.g., for capturing extreme sickling of the cell) to 1 for perfect circle. In implementations, the area and the perimeter are determined by the sample analyzer 310 via processing of the image data captured by the sample camera 308. During experimental analysis of a variety of sample cells, it has been observed that adherent cells typically have a circularity of less than 0.6 and tend to not move during flow of fluid by the sample pump 300 (e.g., by adhering to interior surfaces of the MMM device 102), whereas cells having a circularity of 0.6 to 1 tend to move through the MMM device 102 during operation of the sample pump 300.

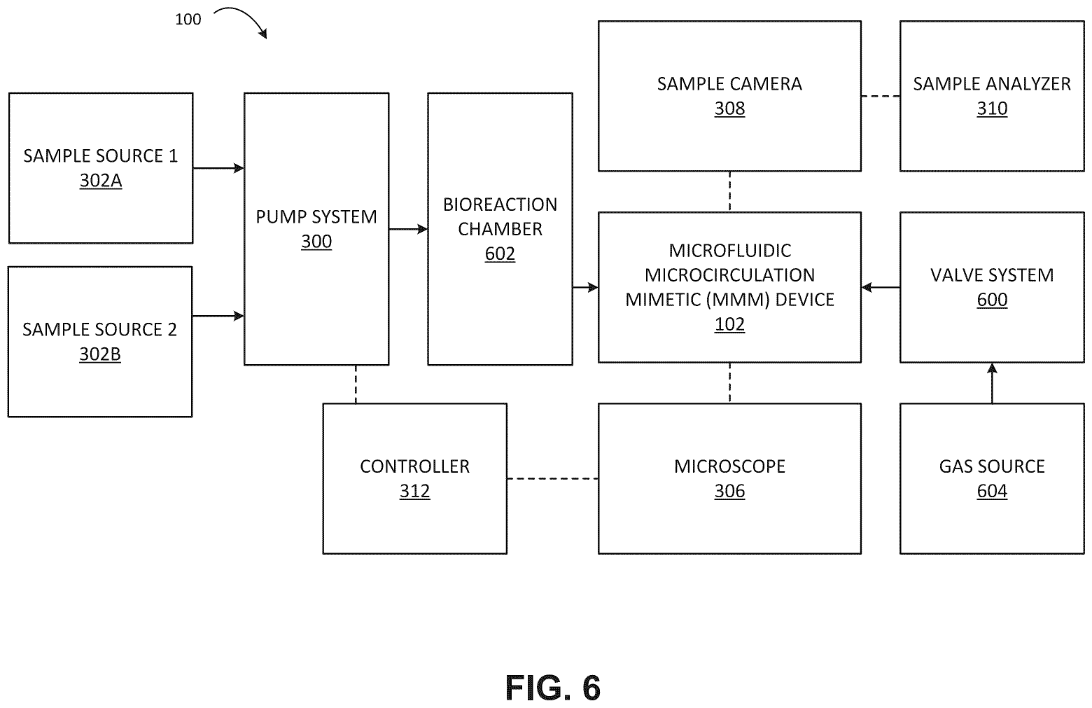

The system 100 can include components to control various operating conditions for the MMM device 102, to modify the composition of samples for introduction to the MMM device 102, or to modify the composition of samples following introduction to the MMM device 102. For example, referring to FIG. 6, the system 100 is shown including a valve system 600 for controlling gas conditions within the MMM device 102 and a bioreaction chamber 602 for interacting multiple cell types prior to introduction to the MMM device 102. The valve system 600 is shown fluidically coupled with a gas source 604 and with the MMM device 102 to introduce one or more gases received from the gas source 604 to the MMM device 102. In implementations, the gas source 604 includes oxygen such that the valve system 600 controls the amount of oxygen within a gas mixture (e.g., including nitrogen and/or other gas(es)) to maintain the MMM device 102 as one or more of a hypoxic oxygen environment, a normoxic oxygen environment, or a hyperoxic oxygen environment. For example, the valve system 600 can be fluidically coupled with the sample to introduce a predetermined amount or volume of oxygen to the sample (e.g., via diffusion, direct introduction, or other technique(s)) prior to introduction of the sample to the MMM device 102. The valve system 600 can include, but is not limited to, one or more rotary selection valves configured to receive fluid lines fluidically coupled with one gas source 604, multiple gas sources 604, vacuums, mixing chambers, or the like, to facilitate preparation of a gaseous environment for transfer to the MMM device 102.

The bioreaction chamber 602 is configured to receive one or more sample sources, chemical sources, reagent sources, solvent sources, or the like, to modify a sample prior to introduction to the MMM device 102. For example, the bioreaction chamber 602 can be fluidically coupled between the MMM device 102 and a plurality of sample sources, chemical source, reagents sources, solvent sources, or the like, or combinations thereof (e.g., supplied by pump system 300, which can include one or more pumps to drive the fluid sources to the biorcaction chamber 602) to mix, interact, or otherwise combine the fluids together for introduction of the mixture to the MMM device 102. For instance, the system 100 is shown with the pump system 300 fluidically coupled with a first sample source 302A and a second sample source 302B and with the bioreaction chamber 602 to direct a first sample and a second sample for mixing in the bioreaction chamber 602. The pump system 300 can include multiple pumps to individually control the flow rates of the first sample and the second sample according to a mixture ratio, which can be defined by the controller 312. In implementations, the bioreaction chamber 602 facilitates visualization and/or quantification of interaction between RBCs and other cell types, drug types, chemical types, or the like through operation of the microscope 306 and/or the sample camera 308. Alternatively or additionally, the system 100 can include the bioreaction chamber 602 to introduce a sample passed through the MMM device 102 to one or more chemicals, reagents, solvents, or the like to observe or quantify interactions subsequent to passage through the MMM device 102.

Electromechanical devices (e.g., electrical motors, servos, actuators, or the like) may be coupled with or embedded within the components of the system 100 to facilitate automated operation via control logic embedded within or externally driving the system 100. The electromechanical devices can be configured to cause movement of devices and fluids according to various procedures, such as the procedures described herein. The system 100 may include or be controlled by a computing system having a processor or other controller configured to execute computer readable program instructions (i.e., the control logic) from a non-transitory carrier medium (e.g., storage medium such as a flash drive, hard disk drive, solid-state disk drive, SD card, optical disk, or the like). The computing system can be connected to various components of the system 100, either by direct connection, or through one or more network connections (e.g., local area networking (LAN), wireless area networking (WAN or WLAN), one or more hub connections (e.g., USB hubs), and so forth). For example, the computing system can be communicatively coupled to the chamber 102, the motor system, valves described herein, pumps described herein, other components described herein, components directing control thereof, or combinations thereof. The program instructions, when executed by the processor or other controller, can cause the computing system to control the system 100 (e.g., control pumps, valves, microscopes, cameras, etc.) according to one or more modes of operation, as described herein.

It should be recognized that the various functions, control operations, processing blocks, or steps described throughout the present disclosure may be carried out by any combination of hardware, software, or firmware. In some embodiments, various steps or functions are carried out by one or more of the following: electronic circuitry, logic gates, multiplexers, a programmable logic device, an application-specific integrated circuit (ASIC), a controller/microcontroller, or a computing system. A computing system may include, but is not limited to, a personal computing system, a mobile computing device, mainframe computing system, workstation, image computer, parallel processor, or any other device known in the art. In general, the term “computing system” is broadly defined to encompass any device having one or more processors or other controllers, which execute instructions from a carrier medium.

Program instructions implementing functions, control operations, processing blocks, or steps, such as those manifested by embodiments described herein, may be transmitted over or stored on carrier medium. The carrier medium may be a transmission medium, such as, but not limited to, a wire, cable, or wireless transmission link. The carrier medium may also include a non-transitory signal bearing medium or storage medium such as, but not limited to, a read-only memory, a random access memory, a magnetic or optical disk, a solid-state or flash memory device, or a magnetic tape.

### Conclusion

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims. It is apparent that various modifications and embodiments of the structural features and/or process operations may be made by those skilled in the art without departing from the scope and spirit of the foregoing disclosure.

1. A system for patient-specific prognoses of blood conditions, comprising:
a microfluidic microcirculation mimetic (MMM) device configured to pass a blood sample therethrough, the MMM device including
an inlet adhesion area configured to receive the blood sample,
a microfluidic channel fluidically coupled with the inlet adhesion area to receive the blood sample from the inlet adhesion area, and
an outlet adhesion area fluidically coupled with an outlet end of the microfluidic channel to receive the blood sample from the microfluidic channel;
a sample camera mounted relative to the MMM device, the sample camera configured to record images of the blood sample as the blood sample at least one of passes through the MMM device or is retained in the MMM device;
a pump configured to introduce the blood sample to the inlet adhesion area and to transfer the blood sample through the MMM device; and
a controller communicatively coupled with the pump, the controller configured to control a pump rate of the pump to establish a flow rate of the blood sample through the MMM device,
wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells.

2. The system of claim 1, wherein the microfluidic channel includes a plurality of serpentine channel portions having no branching paths.

3. The system of claim 2, wherein the microfluidic channel includes no constrictions.

4. The system of claim 2, wherein the microfluidic channel includes one or more constrictions configured to deform at least one blood cell in the blood sample.

5. The system of claim 1, wherein the functionalizable adhesion area includes one or more of poly-D-lysine or poly-L-lysine.

6. The system of claim 1, further comprising a valve system fluidically coupled with the MMM device and configured to fluidically couple with one or more gas sources, the valve system configured to introduce one or more gases from the one or more gas sources to the inlet adhesion area to control a gaseous environment composition within the MMM device.

7. The system of claim 1, further comprising a bioreactor chamber fluidically coupled between the pump system and the MMM device, the bioreactor chamber configured to receive the blood sample and at least one more fluid from the pump system for mixture within the bioreactor chamber to provide a mixed sample prior to introduction of the mixed sample to the MMM device.

8. The system of claim 1, wherein the controller is configured to vary a pump rate of the pump to provide multiple flow rates of the blood sample through the MMM device.

9. The system of claim 8, wherein the controller is configured to establish a first pump rate to introduce the blood sample into the MMM device, to establish a second pump rate to substantially cease flow of the blood sample through the MMM device to permit attachment of blood cells onto one or more internal surfaces of the MMM device, and to incrementally increase flow rate from the second pump rate to subsequently detach cells from the one or more internal surfaces.

10. The system of claim 9, wherein the sample camera is configured to record a time at which one or more of the cells detached from the one or more internal surfaces.

11. The system of claim 10, further comprising a sample analyzer communicatively coupled with the sample camera, the sample analyzer configured to determine a cell detachment force based on a pump rate of the pump system at the time at which one or more of the cells detached from the one or more internal surfaces.

12. A microfluidic microcirculation mimetic (MMM) device configured to receive a blood sample for patient-specific prognoses of blood conditions in the blood sample, the MMM device comprising:
an inlet adhesion area configured to receive the blood sample;
a microfluidic channel fluidically coupled with the inlet adhesion area to receive the blood sample from the inlet adhesion area; and
an outlet adhesion area fluidically coupled with an outlet end of the microfluidic channel to receive the blood sample from the microfluidic channel,
wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells.

13. The system of claim 12, wherein the microfluidic channel includes a plurality of serpentine channel portions having no branching paths.

14. The system of claim 13, wherein the microfluidic channel includes no constrictions.

15. The system of claim 13, wherein the microfluidic channel includes one or more constrictions configured to deform at least one blood cell in the blood sample.

16. The system of claim 12, wherein the functionalizable adhesion area includes one or more of poly-D-lysine or poly-L-lysine.

17. A method for determining a cell detachment force of a red blood cell from a blood sample for providing patient-specific prognoses of blood conditions in the blood sample, the method comprising:
introducing a blood sample at a first flow rate to a microfluidic microcirculation mimetic (MMM) device, the MMM device including
an inlet adhesion area configured to receive the blood sample
a microfluidic channel fluidically coupled with the inlet adhesion area to receive the blood sample from the inlet adhesion area, and
an outlet adhesion area fluidically coupled with an outlet end of the microfluidic channel to receive the blood sample from the microfluidic channel,
wherein at least one of the inlet adhesion area or the outlet adhesion area includes a functionalizable adhesion area including one or more surface treatments of chemicals, the functionalizable adhesion area configured to interact with adherent red blood cells to a greater extent than non-adherent red blood cells based on a morphology of the red blood cells;
ceasing flow of the blood sample to the MMM device to permit attachment of blood cells from the blood sample onto one or more internal surfaces of the MMM device;
incrementally increasing the flow rate of the blood sample to detach cells from the one or more internal surfaces; and
determining a detachment force associated with detachment of the cells from the one or more internal surfaces based on the flow rate at a time at which one or more of the cells detached from the one or more internal surfaces.

18. The method of claim 17, wherein the microfluidic channel includes a plurality of serpentine channel portions having no branching paths.

19. The method of claim 18, wherein the microfluidic channel includes no constrictions.

20. The method of claim 17, wherein the functionalizable adhesion area includes one or more of poly-D-lysine or poly-L-lysine.
